(19) United States
(12) Patent Application Publication
(10) Pub. No.: US 2009/0081314 A1
(43) Pub. Date: Mar. 26, 2009

(54) Glycemic Control, Diabetes Treatment, and Other Treatments with Acetyl Cholinesterase Inhibitors

(76) Inventor: Stephen Wills, Easton, MD (US)

Correspondence Address: JEFFREY B. OSTER, 8339 SE 57TH ST, MERCER ISLAND, WA, 98040, US

(21) Appl. No.: 12/233,522

(22) Filed: Sep. 18, 2008

Related U.S. Application Data
(60) Provisional application No. 60/973,330, filed on Sep. 18, 2007.

(51) Int. Cl. A61K 33/30; A61K 31/445; A61K 31/55; A61P 3/10; A61P 9/04; A61P 31/18; A61P 3/04; A61K 31/27; A61K 31/435

(52) U.S. Cl. 424/643; 514/319; 514/215; 514/490; 514/297; 514/290

(57) ABSTRACT

There is disclosed a method for glycemic control of a patient having a disease selected from the group consisting of type 1 diabetes mellitus, type 2 diabetes mellitus, impaired glucose tolerance, hyperglycemia, and postprandial hyperglycemia, said method comprising administering to a patient in need thereof a pharmaceutical composition comprising an acetyl cholinesterase inhibitor compound. There is further disclosed a method for reducing HbA_{1C} concentrations as a measure of glycemic control, comprising administering to a patient in need thereof a pharmaceutical composition comprising an acetyl cholinesterase inhibitor compound. Lastly, there is disclosed a pharmaceutical formulation for daily administration comprising an acetyl cholinesterase inhibitor, from about 5 mg to about 15 mg of loratadine and optionally from about 5 mg to about 16 mg of elemental zinc.

### CROSS REFERENCE TO RELATED APPLICATION

This patent application claims priority from U.S. Provisional Patent application 60/973,330 filed 18 Sep. 2007.

### TECHNICAL FIELD

The present disclosure provides a method for glycemic control of a patient having a disease selected from the group consisting of type I diabetes mellitus, type 2 diabetes mellitus, impaired glucose tolerance, metabolic syndrome, hyperglycemia, and postprandial hyperglycemia, of for treating a disease selected from the group consisting of coronary artery disease, cephalization of body mass, obesity, various cancers, and HIV and retroviral infections, said method comprising administering to a patient in need thereof a pharmaceutical composition comprising an acetyl cholinesterase inhibitor compound. The present disclosure further provides a method for reducing HbA_{1C} concentrations as a measure of glycemic control, comprising administering to a patient in need thereof a pharmaceutical composition comprising an acetyl cholinesterase inhibitor compound. Lastly, the present disclosure provides a pharmaceutical formulation for daily administration comprising an acetyl cholinesterase inhibitor, from about 5 mg to about 15 mg of loratadine and optionally from about 5 mg to about 16 mg of elemental zinc.

### BACKGROUND

Type-2 diabetes is a carbohydrate metabolism disorder thought to be caused by a combination of hereditary and environmental factors. Individuals afflicted with type-2 diabetes typically demonstrate inadequate secretion or utilization of insulin, excessive urine production, and excessive amounts of sugar in the blood and urine. Established risk, factors for the development of type-2 diabetes include obesity, an unfavorable body fat distribution, impaired glucose tolerance, hyperinsulinemia and insulin resistance. Insulin resistance, at least initially and often throughout the patient's lifetime, fundamentally underlies the pathophysiology of type-2 diabetes and improving insulin sensitivity is one of the primary therapeutic approaches and provides a valuable assessment of this disease state. Obesity, especially visceral obesity, and dyslipidemia have been reported to be associated with most of the type-2 diabetic subjects. They are also the risk factors for developing the disease. One of the treatment goals in diabetes is to prevent chronic complications, which includes aggressive control of obesity, dyslipidemia and hypertension.

Globally, the number of people with diabetes is expected to rise from the current estimate of 150 millions to 220 millions in 2010 and 300 millions in 2025. The prevalence is increasing in the developing countries such as India, particularly in urban areas. The estimated number of diabetes patients in India was 19.4 million in 1995 and are expected to be 57.2 million in 2025 (W.H.O). In the United States, it is estimated that as of 2002, 18.2 million people (6.3% of the total population) were diabetic. Approximately one in every 400-500 children and adolescents has Type-1 diabetes. In the age group of 20 years or older, 18 million (8.7% in men and 9.3% in women) have diabetes. In people above the age of 60 years, 8.6 million (18.3% of all people in this age group) have diabetes.

There are mainly two types of diabetes. In Type-1 diabetes there is decreased insulin production and the circulating insulin level is very low. Type-1 diabetes usually strikes children and young adults, although the disease onset can occur at any age. It accounts for 5-10% of all diagnosed cases of diabetes. Risk factors for Type-1 diabetes may include autoimmune, genetic, and environmental factors.

Type-II diabetes was previously called non-insulin dependent diabetes mellitus (NIDDM) or adult onset diabetes. It usually begins as insulin resistance, a disorder in which the cells of the body fails to respond to insulin properly. As the need for insulin rises, the pancreas gradually loses its ability to produce insulin. Beta cells of pancreatic islets are dysfunctional. Type-II diabetes is associated with older age group, obesity, family history of diabetes, history of gestational diabetes, impaired glucose metabolism, physical inactivity, and race/ethnicity. In recent years, Type-II diabetes is increasingly being diagnosed in children and adolescents. Type II diabetes is the predominant form of diabetes world wide, accounting for 90% of cases globally.

Conventional therapy consists of one or two daily injection of insulin including mixed intermediate and rapid acting insulin daily, with self monitoring of urine and blood glucose. Intensive therapy gives good glycemic control and decreases the risk of retinopathy by 47%, microalbuminuria by 34%, while secondary intervention causes 43% protection. According to the American Diabetes Association's revised guidelines for 2002, the goal of therapy is to achieve average preprandial plasma glucose concentration in the range of 90-130 mg/dl, average bedtime plasma glucose values between 110-150 mg/dl and HbA1C values less than 7%.

In Type II diabetic patients, the major predisposing factor is obesity. Hence treatment is initiated with dietary changes and exercise and if the patient does not improve, drugs are prescribed. Interacting defects in muscle, liver, adipose tissue and pancreas, generate the pathogenic milieu that results in diabetes. Various classes of oral antihyperglycemic agents are currently available that target the different pathologic factors contributing to diabetes, for example, α-glucosidase inhibitors that delay intestinal carbohydrate absorption, biguanidines that target hepatic insulin resistance, insulin secretagogues that increase pancreatic insulin secretion, thiozolidonediones as insulin sensitizers to target adipocyte and muscle insulin resistance, and intestinal lipase inhibitors to inhibit fat absorption and promote weight loss in obese patients. However, none of these address the various aspects of diabetes concurrently, namely, control of blood sugar, regeneration of the beta cells of pancreas resulting in increased secretion of insulin, reducing insulin resistance, and correcting the decreased hepatic glycogen synthesis.

Type 2 diabetes is an increasingly prevalent disease that due to a high frequency of complications leads to a significant reduction of life expectancy. Because of diabetes-associated microvascular complications, type 2 diabetes is currently the most frequent cause of adult-onset loss of vision, renal failure, and amputations in the industrialized world. In addition, the presence of type 2 diabetes is associated with a two to five fold increase in cardiovascular disease risk. After long duration of disease, most patients with type 2 diabetes will eventually fail on oral therapy and become insulin dependent with the necessity for daily injections and multiple daily glucose measurements.

The UKPDS (United Kingdom Prospective Diabetes Study) demonstrated that intensive treatment with metformin, sulfonylureas or insulin resulted in only a limited improvement of glycemic control (difference in HbA1c .about. 0.9%). In addition, even in patients within the intensive treatment arm glycemic control deteriorated significantly over time and this was attributed to deterioration of beta-cell function. Importantly, intensive treatment was not associated with a significant reduction in macrovascular complications, i.e. cardiovascular events.

Glycemic control is set up as a target for treatment of these diabetic patients, and the purposes are to maintain their quality of dairy life (QOL) like healthy people and to ensure their lives like healthy people by maintaining their good state of glycemic control, and furthermore, to prevent development and progression of diabetic microvascular complications (diabetic retinopathy, diabetic nephropathy, diabetic neuropathy and the like) and arteriosclerotic diseases (ischemic heart disease, cerebrovascular disease, arteriosclerosis and the like). HbA_{1C} value is used as a primary indication, and the targeted value is preferably not more than 7% and more preferably less than 6.5%. In addition, a 2 hour value of postprandial plasma glucose and a fasting plasma glucose are used as supportive indications of hemoglobin HA1c (HbA_{1C}) value. Two hundred (200) mg/dL for 2 hour value of postprandial plasma glucose and 100 to 140 mg/dL for fasting plasma glucose are targeted, respectively.

In a recent large-scale clinical study in the UK on type II diabetes, the importance of glycemic control for treatment of diabetes has been confirmed. For example, 0.9% decrease in HbA_{1C} value caused 10% reduction of diabetes-associated mortality. It has been reported that the occurrence of cardiac infarction and microvascular complication notably decreased by 16% and 25%, respectively, and that provides good effects on development and progression of diabetic complications. Furthermore, it has been reported that overt diabetic nephropathy is increasingly frequent with HbA_{1C} value over 7.5% and diabetic retinopathy occurs in high frequency in cases with fasting plasma glucose of 140 mg/dL or more.

Therefore there is an unmet medical need for drugs with a good efficacy with regard to glycemic control, with regard to disease-modifying properties and with regard to reduction of cardiovascular morbidity and mortality while at the same time showing an improved safety profile. Thus, there is a need in the art for a safe and effective treatment for treating, preventing, or reducing the risk of developing diabetes and for increasing glycemic control.

U.S. Patent Application 2005/0049293 describes a method for “reducing insulin resistance” by administering a cholinesterase antagonist. Insulin resistance is described a “reduction in the response to insulin secondary to a failure of HISS (hepatic insulin sensitizing substance) action on glucose disposal.” Therefore, this addresses only type 2 diabetes causation and not the secondary effects of diabetes.

U.S. Patent Application 2005/0129350 describes a method for treating diabetes, wherein diabetes is described as “including prevention or reduction in insulin resistance and/or to treat dementia associated with Abeta protein and neurofibril tangles.” The drugs described in this patent application include “a phenserine compound or a phenserine-like compound.” The published patent application appears to be mere conjecture with no data and no in vitro, no in vivo and no clinical results presented.

### Coronary Artery Disease

Coronary artery disease is the principal cause of death in the United States, Europe and most of Asia. Coronary artery disease is a narrowing of the coronary arteries that supply blood and oxygen to the heart. Coronary disease usually results from the build up of fatty material and plaques (atherosclerosis). As a result of coronary artery stenosis, the flow of blood to the heart can slow or stop. The disease can be characterized by symptoms, including but not limited to, chest pain (stable angina), shortness of breath, atherosclerosis, ischemia/reperfusion, hypertension, restenosis and arterial inflammation.

Coronary artery disease affects the lives of millions of people, and may affect the health of a patient without warning. Detection of coronary artery stenosis involves patient history, physical examination, stress testing and possibly a coronary angiogram. Beyond history and physical examination, the diagnostic technique is associated with significant cost and risk. Although the stress test is the most frequently ordered test to detect possible coronary artery disease, sensitivity and specificity of the stress test vary greatly from 40 percent to 90 percent, depending upon whether there is single or multi-vessel disease.

Despite dramatic advances in the treatment of heart disease over the past three decades, coronary artery disease (CAD) remains the leading cause of death in the Western world (“Mortality from coronary heart disease and acute myocardial infarction” Morbidity & Mortality Weekly Report 50:90-93, 2001). More specifically, while preventative measures and “mechanical” revascularization strategies (angioplasty and bypass surgery) have resulted in five year survival rates in excess of 80% for individuals who are candidates for such therapies, treatment options remain limited when coronary disease has progressed to diffuse, occlusive disease, and/or infarction (American Heart Association, Heart and Stroke Statistical Update, 2003). The two-year survival rate for individuals with such advanced coronary artery disease is as low as 20% (Anyanwu et al. Brit. Med. J. 326:509-510, 2003).

Each year, almost 1.1 million Americans suffer an acute myocardial infarction (American Heart Association, Heart and Stroke Statistical Update, 2003). Early intervention can limit infarct size and improve early survival (Mitchell et al. J. Am. Coll. Cardiol 19:1136-44, 1992; Migrino et al. Circulation 96:116-121, 1997; Boyle et al. Circulation 88:2872-83, 1993). However, 20% of those patients surviving an acute myocardial infarction will develop significant left ventricular dilatation with a left ventricular end-systolic volume index (LVESVI) of less than 60 mL/m². The GUSTO I trial (Migrino et al. Circulation 96:116-121, 1997) documented that left ventricular dilatation following myocardial infarction is an independent and significant predictor of mortality. Therefore, whereas early survival after myocardial infarction may be predicated by the timeliness and adequacy of appropriate reperfusion therapy, long-term prognosis is strongly dependent on subsequent changes in left ventricular geometry and function These are the determinants of congestive heart failure (Mitchell et al. J. Am. Coll. Cardiol. 19:1136-44, 1992; Gheorghiade et al. Circulation 97:282-89, 1998; White et al. Circulation 76(1):44-51, 1987).

Congestive heart failure (CHF), which can result from an acute myocardial infarction, currently affects over 5 million people in the United States (National Heart Lung and Blood Institute National Institutes of Health Data Fact Sheet: congestive heart failure in the United States: A new epidemic, NHLBI web site. www/nhlbi.nih.gov/health/public/heart/other/chf.htm; O'Connell et al. “Economic impact of heart failure in the United States: time for a different approach” J. Heart Lung Transplant. 13:S107-S112, 1994). Medical therapies, despite some progress, still confer only a <50% one-year survival in patients with the most severe clinical manifestations of end-stage CHF (Rose et al. “Long-term use of a left ventricular assist device for end-stage heart failure” NEJM 345(20): 1435-43, 2001). Despite its clinical effectiveness, heart transplantation is a therapy with little epidemiological significance in the fight against heart failure (Taylor et al. J. Heart Lung Transplant. 22(6):616-624, 2003). As a result, cell-based therapies for repair and regeneration of infarcted myocardium have been proposed to treat patients suffering from chronic heart failure (Chiu et al. Ann. Thor. Surg. 60:12-8, 1995; Pagani et al. J. Am. Coll. Cardiol. 41:879-888, 2003; Ghostine et al. Circulation 106:1131-1136, 2002; Dorfman et al. J. Thor. Cardiovasc. Surg. 116:744-51, 1988; Taylor et al. “Regenerating functional myocardium: improved performance after skeletal myoblast transplantation” Nat. Med. 4(8):929-33, 1998; Retuerto et al. J. Thorac. Cardiovasc. Surg. 127:1-11, 2004; Jain et al. Circulation 103:1920-27, 2001; Reinecke et al. Circ. Res. 94(6):e56-60, 2004; McConnell et al. J. Thorac. Cardiovasc. Surg. 2004; Kessler et al. Ann. Rev. Physiol. 61:219-42, 1999; Tse et al. Lancet 361:47-49, 2003; Kamihata et al. Circulation 104:1046-1052, 2001).

Therefore, there is a continuing need in the art for improved pharmacologic agents that can address coronary artery disease. The present disclosure was based upon the surprising discovery of a class of drugs (not associated with coronary artery disease) can indeed significantly improve patient outcomes, in fact better that current standards of care.

### Cephalization of Body Mass/Obesity

Cephalization of body mass refers to a weight or tissue distribution in the human body. Essentially, cephalization of body mass means lessening distribution of fat tissue about the pelvis, abdomen areas and a redistribution of mass for women (often more muscle mass) in the areas of the shoulder girdle and bust. For men the redistribution is more prominent shoulder muscle mass absent gynocomastia. For women, cephalization of body mass generally results in a slimmer waste/hips and a larger and firmer breast.

There is currently no regulatory approved and generally accepted treatment or product for cephalization of body mass, but in conversations with many women in their 30's and 40's indicated a strong desire for such a treatment if effective. Therefore, there is a need (as evidenced by conversations with the attorney writing this patent application and the inventor (both males)) of a desire for such a treatment.

Treatment of obesity means weight loss, primarily fat tissue and not muscle mass. There have been many such treatments for obesity, which generally try to increase metabolism to use more energy or try to limit the intake of food or its absorption from the GI tract. However, none have tried a cholinergic agonist, particularly an acetylcholinesterase inhibitor. Some anti-obesity drugs have severe and often life-threatening side effects (for example, Fen-phen). The drug side effects are often associated with their mechanism of action. In general, stimulants carry a risk of high blood pressure, faster heart rate, palpitations, closed-angle glaucoma, drug addiction, restlessness, agitation, and insomnia.

One drug, Orlistat, blocks absorption of dietary fats, and as a result may cause oily spotting bowel movements, oily stools, stomach pain, and flatulence. A similar medication, designed for patients with Type 2 diabetes, is Acarbose which partially blocks absorption of carbohydrates in the small intestine, and produces similar side effects including stomach pain, and flatulence.

The limitation of drugs for obesity is that we do not fully understand the neural basis of appetite and how to modulate it. Appetite is an important instinct to promote survival. Arguably any drug that would abolish appetite may carry a high mortality risk and may be unsuitable for clinical use. Because the human body uses various chemicals and hormones to protect its stores of fat (a reaction probably useful to our ancestors when food was scarce in the past), there has not yet been found a ‘silver bullet’, or a way to completely circumvent this natural habit of protecting excess food stores. In order to circumvent the number of feedback mechanisms that prevent most monotherapies from producing sustained large amounts of weight loss, it has been hypothesized that combinations of drugs may be more effective by targeting multiple pathways and possibly inhibiting feedback pathways that work to cause a plateau in weight loss. This was evidenced by the success of the combination of phentermine and fenfluramine or dexfenfluramine, popularly referred to phen-fen, in producing significant weight loss but fenfluramine and dexfenfluramine were pulled from the market due to safety fears regarding a potential link to heart valve damage. The damage was found to be a result of activity of fenfluramine and dexfenfluramine at the 5-HT2B serotonin receptor in heart valves. Newer combinations of SSRIs and phentermine, known as phenpro, have been used with equal efficiency as fenphen with no known heart valve damage due to lack of activity at this particular serotonin receptor due to SSRIs.

Other classes of drugs in development include lipase inhibitors, similar to Xenical (Orlistat). Another lipase inhibitor, called GT 389-255, is being developed by Peptimmune (licensed from Genzyme). This is a novel combination of an inhibitor and a polymer designed to bind the undigested triglycerides therefore allowing increased fat excretion without side effects such as oily stools that occur with Xenical. The development seems to be stalled as Phase 1 trials were conducted in 2004 and there has been no further human clinical development since then. Another potential long-term approach to anti-obesity medication is through the development of ribonucleic acid interference (RNAi). Animal studies have illustrated that the deletion of the RIP140 gene in mice by genetic knockout results in the lack of fat accumulation, even when mice are fed a high fat diet. Experiments conducted by Professor Malcolm Parker of Imperial College show that by silencing RIP 140, a nuclear hormone co-repressor which regulates fat accumulation, animal models exhibit a lean profile throughout their life, are resistant to diet-induced obesity, and show an enhanced metabolic rate. Therefore, there is a need to develop established and known drugs for obesity indications because of a known side effect profile.

### Metabolic Syndrome

The criteria or cluster of symptoms for a patient with metabolic syndrome (essentially a pre Type 2 diabetes condition) is symptoms taken from the group of elevated triglycerides, decreased HDL cholesterol, high blood pressure (hypertension), or elevated fasting sugar. Metabolic syndrome is identified by a constellation of central obesity, dyslipidemia (hypertriglyceridemia and low levels of high-density lipoprotein [HDL] cholesterol), elevated blood pressure, and insulin resistance that leads to increased risk of cardiovascular disease and type 2 diabetes. Other ways to characterize metabolic syndrome include: The metabolic syndrome is characterized by a group of metabolic risk factors in one person. They include: Abdominal obesity (excessive fat tissue in and around the abdomen); Atherogenic dyslipidemia (blood fat disorders—high triglycerides, low HDL cholesterol and high LDL cholesterol—that foster plaque buildups in artery walls); Elevated blood pressure; Insulin resistance or glucose intolerance (the body cannot properly use insulin or blood sugar); Prothrombotic state (e.g., high fibrinogen or plasminogen activator inhibitor-1 in the blood); and Proinflammatory state (e.g., elevated C-reactive protein in the blood). A large percentage of the population can be characterized as having metabolic syndrome, leading to diabetes.

### HIV

HIV or human immunodeficiency virus is an infection with a retrovirus that does not have a treatment that can completely cure the infection, only manage it as a chronic disease. The state of HIV infection is often monitored by CD4 counts (the higher the better) and circulating virus (the lower the better).

### Morgellon's Syndrome

Morgellon's Syndrome is characterized by fibers below the skin and generalized skin lesions. Morgellons (also called Morgellons disease or Morgellons syndrome) is a name given in 2002 by biologist Mary Leitao to a condition characterized by a range of cutaneous (skin) symptoms including crawling, biting, and stinging sensations; finding fibers on or under the skin; and persistent skin lesions (e.g., rashes or sores). It is also characterized by skin lesions, joint arthralgia and memory loss. The Centers for Disease Control and Prevention (CDC) states that it is not known at present whether the condition represents a new disease entity, or whether persons who identify themselves as having Morgellons have a common cause for their symptoms, share common risk factors, or are contagious. Although the disease itself and its etiology and diagnosis is relatively controversial, there is not accepted treatment, nor any suggested in recent articles describing it. Therefore, there is a need for a treatment of this disease once it is better characterized and its diagnosis better elucidated.

### Restless Leg Syndrome

This syndrome is cramps and an uncomfortable feeling while in bed. It has been tied to sleep apnea. Restless legs syndrome (RLS) is a neurological condition that is characterized by the irresistible urge to move the legs. In order to be officially diagnosed with RLS, there are four criteria:

(1) A strong urge to move one's legs which one may not be able to resist. The need to move is often accompanied by uncomfortable sensations. Some words used to describe these sensations include: creeping, itching, pulling, creepy-crawly, lugging, or gnawing.

(2) The RLS symptoms start or become worse when one are resting. The longer one rests, the greater the chance the symptoms will occur and the more severe they are likely to be. The RLS symptoms get better when legs are moved. The relief can be complete or only partial but generally starts very soon after starting an activity. Relief persists as long as the motor activity continues.

(3) RLS symptoms are worse in the evening especially when lying down. RLS can also cause difficulty in falling or staying asleep which can be one of the chief complaints of the syndrome. A substantial number of people who have RLS also have periodic limb movements of sleep (PLMS). These are jerks that occur every 20 to 30 seconds on and off throughout the night. This can cause partial awakenings that disrupt sleep.

### Side Effects of Chronic Steroid Use

The side effects of chronic steroid (such as prednisone) use include centripetal obesity (similar to metabolic syndrome), elevated blood sugars (metabolic syndrome), elevated blood pressure (metabolic syndrome) and mania or steroid psychosis.

Acetyl cholinesterase Inhibitors

The current treatment armamentarium for Alzheimer's Disease (AD) consists of four cholinesterase inhibitors and an N-methyl-D-aspartate (NMDA) antagonist. The cholinesterase inhibitors that are approved for use in the United States are donepezil (Aricept®), rivastigmine (Exelon®), galantamine (Peminyl®) and tacrine (Cognex®), with the latter being rarely used. Findings of controlled trials demonstrated that the cholinesterase inhibitors preserve levels of acetylcholine in the brain and may provide modest, transient improvement in cognitive and behavioral symptoms. Acetylcholine production declines progressively in AD, eventually reaching the point where cholinesterase inhibition has no benefit. The findings of short-term studies demonstrated that cholinesterase inhibitors are superior to placebo on measures of global and cognitive function in patients with mild-to-moderately severe AD. However, it is not universally agreed that these changes translate into positive outcomes such as maintenance of activities of daily living, reduced caregiver burden and delayed nursing home.

Acetyl cholinesterase inhibitors (such as, donepezil and rivastigmine) have been shown to improve blood flow to CNS structures on PET scanning. Choline esters have demonstrated the ability to improve cutaneous blood flow as well as increase skin temperature when administered via ionophoresis. Acetyl cholinesterase inhibitors are cholinomimetic agents inhibiting the break down of acetylcholine and therefore facilitating cholinergic functions. Treatment with an acetyl cholinesterase inhibitor has a general distribution throughout the body as demonstrated in the side effect profile. The net effect of treatment with an acetyl cholinesterase inhibitor is to enhance cholinergic functions in the body. The parasympathetic nervous system makes use of cholinergic transmission to facilitate homeostatic function in the body, resulting in lowering blood pressure decreasing heart rate and facilitating gastrointestinal transit and many other cholinergic activities related to the parasympathetic nervous system. Neuronal and non-neuronal acetylcholine receptors exist within the intima of blood vessels. This would accounts for the dilitation of blood vessels and lowering of blood pressure associated with administration of acetylcholinisterase inhibitors.

### SUMMARY

The present disclosure provides a method for glycemic control of a patient having a disease selected from the group consisting of type I diabetes mellitus, type 2 diabetes mellitus, impaired glucose tolerance, hyperglycemia, and postprandial hyperglycemia, said method comprising administering to a patient in need thereof a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the weekly dose of donepezil is from about 15 mg to about 60 mg taken two to three times per week. The daily dosage of galantamine is from about 16 mg to about 32 mg, and the daily dosage of rivastigmine is from about 3 mg to about 9 mg. Preferably, the acetyl cholinesterase inhibitor compound further comprises from about 10 mg to about 50 mg per day dose of metaclopramide.

The present disclosure provides a method for reducing HbA_{1C} concentrations as a measure of glycemic control, comprising administering to a patient in need thereof a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the weekly dose of donepezil is from about 15 mg to about 60 mg taken two to three times per week. The daily dosage of galantamine is from about 16 mg to about 32 mg, and the daily dosage of rivastigmine is from about 3 mg to about 9 mg.

The present disclosure further provides a pharmaceutical formulation for daily administration comprising an acetyl cholinesterase inhibitor, from about 5 mg to about 15 mg of loratadine, optionally from about 5 mg to about 16 mg of elemental zinc, and optionally, from about 10 mg to about 50 mg per day dose of metaclopramide. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the daily dose of loratadine is from about 8 μg to about 12 mg. Preferably, the elemental zinc daily does is from about 8 mg to about 50 mg.

The present disclosure provides a method for treating diabetes comprising administering a formulation comprising an acetyl cholinesterase inhibitor, from about 5 mg to about 15 mg of loratadine, optionally from about 5 mg to about 16 mg of elemental zinc, and optionally from about 10 mg to about 50 mg per day dose of metaclopramide. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the daily dose of loratadine is from about 8 mg to about 12 mg. Preferably, the elemental zinc daily does is from about 8 mg to about 50 mg.

The present disclosure further provides a method for treating BPH (benign prostatic hypertrophy) by alleviating urinary retention comprising administering to a patient in need thereof a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure further provides a method for promoting wound healing by improving vascular insufficiency, comprising administering to a patient in need thereof a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure provides a method for treating diabetes comprising administering a formulation comprising an acetyl cholinesterase inhibitor and from about 200 mg to about 600 mg of metformin (once or twice daily). Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the twice daily dose of metformin is from about 400 mg to about 1000 mg.

The present disclosure provides a method for treating diabetes, including long term complications of diabetes and glycemic control, comprising administering a formulation comprising an acetyl cholinesterase inhibitor, from about 200 mg to about 1000 mg of metformin (once or twice daily), and optionally and optionally from about 10 mg to about 50 mg per day dose of metaclopramide. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the twice daily dose of metformin is from about 400 mg to about 1000 mg.

The present disclosure further provides a method for reducing necessary doses of insulin or other glycemic control medications comprising administering a formulation comprising an acetyl cholinesterase inhibitor and from about 200 mg to about 600 mg of metformin (once or twice daily). Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the twice daily dose of metformin is from about 400 mg to about 600 mg.

The present disclosure further provides a method for treating cancer by trophic stimulation of neoplastic cancer cells to promote normal growth and function, comprising administering to a patient in need thereof a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure provides a method for treating various forms of coronary artery disease, congestive heart failure and cardiomyopathy in a patient comprising administering to a patient a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure further provides a pharmaceutical formulation for daily administration comprising an acetyl cholinesterase inhibitor, from about 5 mg to about 15 mg of loratadine and optionally from about 5 mg to about 16 mg of elemental zinc. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the daily dose of loratadine is from about 8 mg to about 12 mg. Preferably, the elemental zinc daily does is from about 8 mg to about 50 mg.

The present disclosure provides a method for treating coronary artery disease comprising administering a formulation comprising an acetyl cholinesterase inhibitor, from about 5 mg to about 15 mg of loratadine and optionally from about 5 mg to about 16 mg of elemental zinc. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the daily dose of loratadine is from about 8 mg to about 12 mg. Preferably, the elemental zinc daily does is from about 8 mg to about 50 mg.

The present disclosure provides a method for treating obesity and promoting cephalizalion of body mass, comprising administering to a patient a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure provides a method for treating metabolic syndrome to prevent its progression to diabetes, comprising administering to a patient a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure provides a method for treating HIV infection in an infected patient, comprising administering to the infected patient a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure provides a method for treating the side effects of corticosteroid administration or restless leg syndrome comprising administering to a patient taking a corticosteroid, a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 μg, and the daily dosage of rivastigmine is from about 3 mg to about 9 mg.

The present disclosure provides a method for treating morgellons syndrome comprising administering to a patient a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

The present disclosure provides a method for treating morgellons syndrome comprising administering to a patient a pharmaceutical composition comprising an acetyl cholinesterase inhibitor compound. Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg.

### DETAILED DESCRIPTION

### Acetyl Cholinesterase Inhibitors

Acetyl cholinesterase inhibitors increase the amount of neurotransmitter acetylcholine at the nerve terminal by decreasing its breakdown by the enzyme cholinesterase. European Patent 0296560 discloses a number of compounds indicated as acetyl cholinesterase inhibitors useful in the treatment of Alzheimer's disease. (1-benzyl-4->(5,6-dimethoxy-1-indanon)-2-yl methylpiperidine, is also known as donepezil, E-2020 and Aricept®. Suitable doses of the acetyl cholinesterase inhibitor compounds are indicated to be in the range 0.1 to 300 mg, preferably 1 to 100 mg, per adult per day. Examples of active agents for amyloid-related disorders are doxorubicin, galantamine, tacrine (Cognex), metrifonate, rivastigmine, selegiline, physostigmine, donepezil (Aricept), milameline, xanomeline, saeluzole, acetyl-L-camitine, idebenone, ENA-713, memric, quetiapine, neurestrol and neuromidal. Preferably, the acetyl cholinesterase inhibitor or butylcholinesterase inhibitor is selected from donepezil (Aricept), tacrine (Cognex) rivastigmine (Exelon), physostigmine (Synapton), galanthamine (Reminyl), metrifonate (Promem), quilostigmine, tolserine, thiatolserine, cymserine, thiacymserine, neostigmine, eseroline, zifrosilone, mestinon, huperzine A, phenserine, and icopezil or a pharmaceutically acceptable salt of one of the foregoing compounds.

Acetyl cholinesterase inhibitors are typically administered as a pharmaceutical composition that comprises the acetyl cholinesterase inhibitor that is greater than 95% and preferably greater than 99% pure by weight and one or more excipients, diluents or other inert ingredients commonly found in pharmaceutical compositions. Thus, any acetyl cholinesterase inhibitor that are natural products, i.e., produced in nature, are isolated and purified or produced synthetically before being used in the disclosed method.

### Loratadine

Loratadine is a tricyclic antihistamine, which selectively antagonizes peripheral histamine H₁-receptors. Loratadine is named as ethyl 4-8-chloro-5,6-dihydro-11-H-benzo[5,6]cyclohepta-[1,2-b]-pyridin-11-ylidene)-1-piperidinecarboxylate. One active metabolite of loratadine is known as descarboethoxyloratadine. The metabolite may be prepared by removal of the carboethoxy moiety according to methods known to those skilled in the art.

Loratadine and methods for making loratadine are disclosed in U.S. Pat. No. 4,282,233, the disclosure of which is hereby incorporated by reference. The pharmacokinetics of loratadine is discussed in J. Clin. Pharmacol. 1987; 27:530-533 and J. Clin. Pharmacol. 1987; 27:694-698. The starting materials and reagents for the above described compound, is also readily available or can be easily synthesized by those skilled in the art using conventional methods of organic synthesis.

Loratadine and descarboethoxyloratadine are basic and they form salts with pharmaceutically acceptable anions. All such salts are within the scope of this disclosure and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

### Zinc

Zinc formulations are generally available as a zinc sulfate salt. Standard daily dose is 50 mg.

### Metformin

Metformin has trade names Glucophage, Diabex, Diaformin, Fortaret, Riomet, Glumetza, Cidophage and others. Metformin is an anti-diabetic drug from the biguanide class of oral antihyperglycemic agents. The main use for metformin is in the treatment of diabetes mellitus type 2. It is also being used increasingly in polycystic ovarian syndrome (PCOS), non-alcoholic fatty liver disease (NAFLD) and premature puberty. The benefit of metformin in NAFLD has not been extensively studied and may be only temporary.

Metformin reduces cardiovascular complications of diabetes as shown in a study of overweight patients with diabetes. Metformin monotherapy will not induce hypoglycemia. Hypoglycemia during intense exercise has been documented.

The mechanism of action of metformin is uncertain despite its therapeutic benefits. Its mode of action appears to be reduction of hepatic neogenesis, decreased absorption of glucose from the gastrointestinal tract, and increased insulin sensitivity. The ‘average’ person with type 2 diabetes has three times the normal rate of gluconeogenesis; metformin treatment reduces this by over one third. It has also been shown to decrease intestinal absorption of glucose, and may also improve insulin sensitivity by increasing peripheral glucose uptake and utilization, although such an effect will occur nonspecifically following the lowering of glucose levels, regardless of how this lowering was achieved.

An “effective amount” of a compound is a quantity which, when administered to a subject in need of treatment, improves the prognosis of the subject, e.g., delays the onset of and/or reduces the severity of one or more of the subject's symptoms associated with condition being treated. The amount of the acetyl cholinesterase inhibitor to be administered to a subject will depend on the particular disease, the mode of administration, the bioavailability of the acetyl cholinesterase inhibitor and the characteristics of the subject, such as general health, other diseases, age, sex, genotype, body weight and tolerance to drugs. The skilled artisan will be able to determine appropriate dosages depending on these and other factors. Effective amounts of a pharmaceutically acceptable acetyl cholinesterase inhibitor typically ranges between about 0.1 mg/kg body weight per day and about 1000 mg/kg body weight per day, and preferably between 1 mg/kg body weight per day and 100 mg/kg body weight per day.

The route of administration of the acetyl cholinesterase inhibitor depends on the condition to be treated. For the disclosed treatments of glycemic control and reduction of blood hemoglobin A1c, preferred routes of administration are anything that can provide a systemic concentration of the acetyl cholinesterase inhibitor, including, but not limited to oral, injection (iv, sc, im), transdermal, intranasal, and inhalation. The route of administration and the dosage of the acetyl cholinesterase inhibitor to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Thus, depending on the condition, the acetyl cholinesterase inhibitor can be administered orally, parenterally, intranasally, vaginally, rectally, lingually, sublingually, bucally, and intrabuccaly to the patient.

Accordingly, acetyl cholinesterase inhibitor compositions designed for oral, lingual, sublingual, buccal and intrabuccal administration can be made, for example with an inert diluent or with an edible carrier. The compositions may be enclosed in gelatin capsules or compressed into tablets. For the purpose of oral therapeutic administration, the pharmaceutical compositions of the present disclosure may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

Acetyl cholinesterase inhibitor compositions can be administered parenterally such as for example, by intravenous, intramuscular, intrathecal or subcutaneous injection. Parenteral administration can be accomplished by incorporating the cholinergic agonist compositions of the present invention into a solution or suspension. Such solutions or suspensions may also include sterile diluents such as water for injection; saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the acetyl cholinesterase inhibitor in the glycerin, mixing the heated glycerin after which purified water may be added, and pouring the hot mixture into a suppository mold.

The present disclosure includes nasally administering to the subject an effective amount of the acetyl cholinesterase inhibitor. Nasally administering or nasal administration includes administering the acetyl cholinesterase inhibitor to the mucous membranes of the nasal passage or nasal cavity of the patient. Pharmaceutical compositions for nasal administration of an acetyl cholinesterase inhibitor include therapeutically effective amounts of the acetyl cholinesterase inhibitor prepared by well-known methods to be administered, for example, as a nasal spray, nasal drop, suspension, gel, ointment, cream or powder. Administration of the acetyl cholinesterase inhibitor may also take place using a nasal tampon or nasal sponge.

The acetyl cholinesterase inhibitor can be administered alone (as a monotherapy) or in combination with one or more other pharmaceutically active agents that are effective against the condition being treated. However, the combination therapy does not include an acetyl cholinesterase reactivator, as that the term is used in U.S. Pat. No. 5,981,549, the entire teachings of which are incorporated by reference. For example, an acetyl cholinesterase inhibitor compound can be administered in combination with an acetylcholine receptor agonist (particularly alpha 7 specific agonists and muscarinic receptor agonists that penetrate the blood brain barrier, see, for example, U.S. Pat. No. 6,610,713 and WO 03/072135 and U.S. Ser. No. 10/729,427, filed Dec. 5, 2003—the entire teachings of these three publications are incorporated herein by reference). e.g., anti-microbials, anti-inflammatory agents, analgesics, anti-viral agents, anti-fungals, anti-histamines and the like.

The potential risks and side effects from treatment with an acetyl cholinesterase inhibitor are minimal based upon years of clinical experience using three acetyl cholinesterase inhibitors for treating Alzheimer's disease. The cost saving associated with use of an acetyl cholinesterase inhibitor, relative to the cost of glycemic control agents and nursing care, are readily apparent. Patient compliance with treatment was remarkably good. The incidence of adverse side effects was minimal and did not present an obstacle to treatment with an acetyl cholinesterase inhibitor.

The process of ischemia related to diabetes takes place gradually and relentlessly over time. The incidence of amputation of the contralateral extremity following loss of one limb approaches 50% over the next five years. Patients and physicians are aware of the insidious signs and symptoms that include dystrophic changes in the skin and nails, vascular edema, claudication, and rest pain that occur. Blood flow at the surface level of the skin is most vulnerable. The first appearance of vascular insufficiency is noted in superficial ulceration of the skin. This early sign of ischemia is consistent with microvascular disease and compromise of the very small arterioles and perforators of the skin. Vascular surgical procedures are successful in improving blood flow in the larger arteries. This improved flow increases the fluid pressure that impacts the end arterioles. The improvement in arteriole pressure increases the circulation within the end arterioles and capillaries at the expense of increased edema. The key elements of oxygen and nutrients delivered to the tissue and cells occurs invariably at the microvascular level appears regulated by both neuronal and non-neuronal acetylcholine receptors located in the intima of capillaries and end arterioles. When dilatation of the microvascular circulation is improved delivery of oxygen and nutrients to the cells and tissues occurs with greater facility relieving ischemia. A critical part of the circulation occurs at the microvascular level where exchange of oxygen and nutrients occurs. Simultaneous with delivery of oxygen and nutrients the waste products of metabolism are removed, including carbon dioxide and nitrogenous by products of metabolism.

### Corticosteroid Side Effects

Short-term side-effects of glucocorticoids including prednisone, include high blood glucose levels especially in patients that already have diabetes: mellitus or are on other medications that increase blood glucose (such as tacrolimus), and mineralocorticoid effects such as fluid retention. Additional short-term side-effects include insomnia, euphoria, and, mania. Long-term side-effects include Cushing's syndrome, weight gain, osteoporosis, glaucoma, type II diabetes mellitus, and depression upon withdrawal. The present disclosure found that concomitant treatment with an acetyl cholinesterase inhibitor was able to delay and diminish the well known side effects of glucocorticoids, including prednisone. Particularly noticed were that the rise on blood glucose did not happen, and there was no weight gain.

The present disclosure provides clinical evidence of the use of an acetyl cholinesterase inhibitor for glycemic control, particularly as measured by hemoglobin A1c (HbA_{1C}) levels. Therefore, there is disclosed a method for providing glycemic control in diabetic or pre-diabetic individuals comprising administering an effective amount of an acetyl cholinesterase inhibitor. While glycemic control has historically been accomplished through the administration of insulin to replace the insulin no longer produced in Type I diabetics or improve the bodies response to insulin produced in Type 2 diabetics, the open label patient study reported herein shows that the administration of an acetyl cholinesterase inhibitor significantly improved glycemic control, to the point of providing a reduction in the need for insulin or in the amounts of insulin needed.

Mr. H. D. was a 59 year old male with history of diabetes mellitus, peripheral vascular disease status post left below knee amputation. The patient had been fitted with an insulin pump for management of his blood sugars. On the average he required approximately 32 units of insulin daily for glycemic control of blood glucose levels in the 100-125 mg % range. He suffered from food drop on the intact right lower extremity requiring AFO (Ankle foot orthosis). The patient was started of Aricept (donepezil) 5 mg orally daily. After one month of donepezil treatment his insulin requirement for 24 hour period decreased consistently to less the 20 units. After an additional month of treatment his foot drop resolved and his daily insulin requirement decreased to approximately 12 units over a 24 hour period. He suffered no symptoms of diarrhea, nausea or polyuria.

Ms. Y. D. was a 79 year old female with history of adult onset diabetes and bilateral lower extremity lymphedema and hypertension. Upon admission to a rehabilitation facility her blood glucose control was managed with prandin 2 mg orally before meals. Her blood sugar control was in the range of 100-120 mg % consistently on this regimen. Patient was started on Aricept (donepezil) 5 mg orally daily. After 3 days the preprandial blood sugars were consistently less than 80 mg %. She had one episode of a blood sugar at lunch of less than 50 mg %. Her prandin was discontinued completely. Her blood sugars remained consistently less than 110 mg % on an 1800 ADA diet. Over the next month there was a notable decline in the lymphedema in her lower extremities. Her diet was liberalized to no concentrated sweets with similar control of blood glucose levels.

Mr. J. C. was a 65 year old male with a history of adult onset diabetes meillitus, hypertension and end stage renal disease on hemodialysis. He was admitted to a rehabilitation facility following left below knee amputation. Upon admission patient was started on Aricept (donepezil) 5 mg daily. His admission HbA_{1C} was 6.4. After one week of donepezil 5 mg orally daily his dose was increased to 10 mg orally daily. Follow up HbA_{1C} was measured two weeks after beginning donepezil. The HbA_{1C} after two weeks of treatment with donepezil was 5.7. Glycemic control was better corresponding to the lower HbA_{1C} value. He was discharged on 10 mg orally of donepezil daily for one month. Subsequent value for the HbA_{1C} was 4.6. The patient suffered no untoward side effects from treatment with donepezil.

Mr. L. S. was a 53 year old male with a history of right below knee amputation secondary to peripheral vascular disease. His blood sugar control was accomplished with a split dose of Humulin N 25 units in the morning and 40 units in the evening. Mr. L. S. was started on Aricept (donepezil) 5 mg orally daily. After less than one month his dose of insulin had to be adjusted secondary to low fasting and 4 PM blood sugars. The dose of Humulin N was subsequently reduced from 25 to 10 units in the AM and the evening dose of Humulin reduced from 40 to 20 units. His glycemic control was equal and slightly better with the lower dose of insulin while taking 5 mg of donepezil daily with adverse side effects.

Mr. J. S. was a 61 year old male with history of insulin dependent diabetes mellitus, atrial fibrillation, hypertension, chronic renal insufficiency and diabetic polyneuropathy. His hemoglobin A1C was 11.8 as initially measured. His blood sugar control was managed with Lantus insulin 40 units at bedtime. He was admitted to a rehabilitation hospital for lower extremity weakness secondary to diabetic polyneuropathy. Mr. J. S. was started on donepezil 5 mg orally daily for one week. The dose was titrated upward to 10 mg orally daily after one week when no adverse side effects were noted. His strength improved as well as glycemic control on his original dose of 40 mg of Lantus at bedtime. His BUN (blood urea nitrogen) and creatinine on admission was 38 and 2.0 respectively. The patient continued to receive donepezil 10 mg orally daily for his diabetic polyneuropathy. His HbA_{1C} was measured approximately five weeks later and found to be 9.1 (a reduction from his initial 11.8 reading) An electrocardiogram was obtained demonstrating normal sinus rhythm. Improved glycemic control was obtained as evidenced by the lowering of the HbA_{1C} after 5 weeks of treatment with donepezil. There was significant improvement in the creatinine to 1.2 over the same period of time.

Ms. S. H. was a 73 year old female with a history of Insulin Dependent Diabetes Mellitus admitted to a rehabilitation facility following lumbar laminectomy for stenosis. Upon admission her diabetic control was managed with 12 units of Lanuts (glargine) insulin at bedtime. Her HbA_{1C} was measured on admission and found to be 8.8, indicating poor blood sugar management. The patient was started on Aricept (donepezil) 5 mg orally daily. The patient tolerated the dose of 5 mg orally of donepezil daily and after one week the dose was increased to 10 mg orally at bedtime. The HbA_{1C} was measured one month later and found to have improved to 5.8. During this time her requirements for insulin decreased and she no longer required lantus insulin for control of her blood sugars. When she was started on donepezil the lantus was discontinued and amaryl 4 mg and metformin 500 mg twice daily was begun. Within 48 hours of increasing the dose of donepezil to 10 mg patient discontinued amaryl and was maintained on metformin 500 mg orally twice daily. Over the next two week the dose of metformin was discontinued while maintaining blood glucose values less than 120 mg %. Patient was maintained on an 1800 cal ADA diet. Patient suffered no untoward side effect from treatment with donepezil.

Mr. M. M. was an 86 year old male with a history of adult onset diabetes mellitus treated with lantus (glargine) 5 units at bedtime. The patient was involved in a motor vehicle accident sustaining trauma to the left hemithorax. Upon admission to a rehabilitation facility the patient's hemoglobin A1C was measured at 5.8. The patient was begun on donepezil 5 mg at bedtime. He was treated with this dose for 10 days and the HbA_{1C} level was measured at 5.3. The dose of donepezil was increased to 10 mg orally daily for the next month. His blood sugar control was assessed one month later with an HbA_{1C}. The HbA_{1C} value was 5.0 while at the same time his evening dose of glargine insulin was discontinued. The patient-suffered no negative side effects He reported decreased dyspnea and improved gastrointestinal symptoms while on a daily regimen of donepezil 10 mg at bedtime.

Mr. C. W. was a 63 year old male with history of adult onset diabetes mellitus treated with micronase 5 mg orally daily. He underwent right below knee amputation secondary to vascular insufficiency. Post op his blood glucose control continued to require micronase 5 mg orally for management. Mr. C. W. was begun on Aricept (donepezil) 5 mg orally daily. Over the next 7 days his micronase was discontinued secondary to fasting blood glucose levels less than 90 mg %. After one week of treatment with donepezil 5 mg orally daily the dose was increased to 10 mg. All oral hypoglycemic agents were discontinued secondary to low fasting blood glucoses and control of his other measured blood glucoses less than 120 mg %. The patient suffered no untoward side effects from treatment with donepezil.

Mr. Q. P. was a 43 year old male with history of right below knee amputation, and transmetatarsal amputation. His blood glucose control consisted of 35 units of lantus insulin at bedtime and occasional sliding scale coverage for blood sugars greater than 200 mg %. Mr. Q. P. was started on donepezil 5 mg orally daily. Over the course of one month his insulin requirements decreased to 25 units at bedtime secondary to fasting blood glucoses less than 80 mg %. After one month of treatment with 5 mg orally of donepezil daily the dose was increased to 10 mg daily. His insulin requirements further decreased to 12 units at bedtime secondary to fasting blood glucoses less than 80 mg %. The patient suffered no negative side effects from treatment with donepezil. Over the same period of time his HbA_{1C} improved from 7.3 to a value of 5.8.

Ms. B. W. was a 71 year old female with history of adult diabetes mellitus treated with Avandia 4 mg daily and Prandin 1 mg before meals. Her HbA_{1C} was measured at 9.1 prior to beginning donepezil. After one month of treatment with donepezil 5 mg orally daily at bedtime she no longer required prandin for blood sugar control. Follow up hemoglobin A₁C measured one month later was 6.8. The patient suffered no negative side effects from treatment with donepezil and reported a diminution in painful symptoms of diabetic polyneuropathy.

Ms. E. S. was a 73 year old female with a history of adult onset diabetes mellitus, and chronic renal insufficiency. Prior to starting Aricept (donepezil) 5 mg her HbA_{1C} was measured at 9.3 and her BUN and creatinine were 40 and 1.8 respectively. Her diabetes was managed with glyburide 5 mg orally daily. The patient was begun on Aricept (donepezil) 5 mg daily. After one week of therapy glyburide was discontinued secondary to fasting blood sugars less than 100 mg %. Follow up measurement of HbA_{1C} two months later revealed a value of 6.3. Her BUN and creatinine were improved to 24 and 1.1. She suffered no adverse effects from treatment with donepezil.

Mr. B. L. was a 55 year old male with end stage renal disease, diabetes mellitus, and a right below knee amputation. His blood sugar control was managed with glipizide 5 mg orally daily. He reported blood sugars consistently 150-200 mg % on this regimen while receiving hemodialysis three times a week. He consistently required ultrafiltration of 5-6 liters three times weekly. The patient was started on Aricept (donepezil) 5 mg daily. He was able to discontinue glipizide within the first week. His HbA_{1C} was 8.2 measured prior to treatment with donepezil. Over the next two months patient noted improved appetite, weight gain in the absence of oral hypoglycemic agents. He also reported urine production and commented that his ultrafiltration was consistently less than 2 liter per dialysis. Follow up HbA_{1C} was measured at 5.9 after two months of treatment with donepezil. His fasting blood sugars were consistently less than 100 mg % following treatment with donepezil.

Mr. J. R. was a 70 year old male with history of left below knee amputation admitted to a rehabilitation hospital for therapy needs. He was noted to have renal insufficiency with a BUN of 38 and creatinine of 1.6 on admission. His insulin requirements upon admission were a 70/30 mix given twice daily in doses of 1.7 units in the morning and 15 units in the evening. Endocrine consultation recommended Lantus 30 units at bedtime and 8 units of Novolog before meals. His HbA_{1C} was 9.5 on admission. The patient was started on donepezil 5 mg daily. Over the next 4 days the pre-prandial coverage was reduced to 4 units and the evening dose of lantus insulin decreased to 20 units as a result of fasting blood sugars less than 100 mg %. The patient continued the same dose of donepezil (Aricept) and over the next week the pre prandial coverage was eliminated and the evening dose of lantus reduced further to 10 units secondary to fasting blood sugars less than 100 mg %. The edema in his intact lower extremity was eliminated. Follow up HbA_{1C} measured one month post donepezil therapy was 6.8 and his daily dose of Lantus insulin was further reduced to 5 units at bedtime. The patient suffered no adverse side effect from treatment. A secondary benefit from treatment included improvement in renal function with a BUN and creatinine measured at 21 and 0.9 respectively.

Dr. W.W. was a 66 year old male with history of post polio syndrome, adult onset diabetes mellitus, and congestive heart failure. He had complaints of diabetic polyneuropathy and was started on donepezil 5 mg orally daily. His HbA_{1C} measured prior to starting donepezil was 6.4. His blood glucose management consisted of glipizide 5 mg daily and sliding scale novolog insulin coverage before meals. The patient was also taking furosemide 40 mg daily for management of dependent edema and congestive heart failure. After one month of treatment with donepezil 5 mg daily the patient's pre prandial insulin coverage was eliminated and the daily dose of furosemide significantly reduced to 20 mg every other day. His HbA_{1C} one month later following treatment with donepezil was 5.5. His symptoms of painful diabetic polyneuropathy were significantly reduced without any negative side effects.

Ms. M. F. was a 67 year old female with history of hypertension, adult onset diabetes mellitus admitted to a rehabilitation hospital following right total knee replacement. Her admission HbA_{1C} was 11.5. Her diabetic management included the following oral hypoglycemic agents, poiglitazone 30 mg daily, metformin 500 mg orally twice daily, and glipizide 5 mg orally daily. The patient was started on Aricept (donepezil) 5 mg orally daily. Her fasting blood sugars were noted to be less than 100 mg %, necessitating reduced dose of poiglitazone during the first week of treatment. The patient suffered no episodes of hypoglycemia but over the next 7 days blood sugars less than 111 mg % prompted discontinuation of glipizide in the morning. Follow up HbA_{1C} measured 4 weeks following the start of donepezil was 7.9. The patient experienced no adverse side effects with donepezil.

The present disclosure provides clinical evidence of the use of an acetyl cholinesterase inhibitor for treating coronary artery diseases including heart disease.

Mr. R. D. is a 64 year old male with a history of right above knee amputation, and severe coronary artery disease with pump failure. Patient required daily diuretic therapy 20 mg furosemide twice a day and lisinopril 10 mg daily. His blood pressure was consistently 80/50 with exacerbations of dyspnea with recumbency. He required long acting nitrates along with his blood pressure medications to prevent daily dyspnea on exertion. Patient was started on donepezil (Aricept®) 5 mg daily. Over the next two weeks he reported no shortness of breath and his blood pressure improved to 100/70. His diuretic dose was reduced to every day and follow up one week later revealed no symptoms of shortness of breath. His lisinopril was decreased to 5 mg daily and over the next week his blood pressure improved to 110/70 with no symptoms of dyspnea. His long acting nitrates were discontinued. Eventually his lisinopril was discontinued. After two weeks his blood pressure improved to 120/70. He had no shortness of breath. His endurance improved for gait with his prosthesis. He experienced no symptoms of congestive failure.

During a lapse in follow up visit, he failed to continue his donepezil treatment. This resulted in symptoms of congestive failure with dyspnea in supine position. His blood pressure had deteriorated to 90/60 with symptoms of unstable angina and increased need for sublingual nitroglycerin. Aricept® (donepezil) was restarted and his symptoms resolved over 2448 hours. In the face of cholinesterase inhibitor treatment, the patient was able to discontinue long acting nitrates, diuretics and angiotensin converting enzyme inhibitors while maintaining normal blood pressure, no symptoms of angina, and no symptoms of congestive failure.

Ms. P. H. is a 62 year old female with history of stroke with right sided weakness, severe coronary disease, and congestive failure. She was admitted to a rehabilitation facility status post evacuation of large left sided pleural effusion. Upon admission the patient required diuretics for treatment of her congestive failure coupled with midodrine to maintain blood pressure, but persisted with dyspnea in supine position. The admissions dose of midodrine was 10 mg three times daily and dose of furosemide (diuretic) was 40 mg daily. Despite midodrine, patient blood pressure was 85/60 with persistent dyspnea. Patient was started on Aricept (donepezil) 5 mg daily. Over the next 5 days her dose of midodrine was decreased to 5 mg three times daily, while her blood pressure improved to 100/60. The lower extremity edema was improving along with her pulmonary symptoms allowing for a reduction in her diuretic to 20 mg daily. Over the ensuing week the symptoms of congestive heart failure improved and her blood pressure was consistently 110/60. Midodrine was discontinued completely with no ill effects as regards her blood pressure. The diuretic was further reduced to every other day with continued improved in her cardiac condition. After one month of therapy all diuretic therapy was discontinued. Her blood pressure was 120/70 in the absence of congestive failure, angina, or hypotension. She suffered no untoward side effect from treatment and the right sided weakness in her arm and leg improved allowing gait training.

Mr. M. M. is a 63 year old male with right below knee amputation, severe coronary artery disease with pump failure and end stage renal disease. Patient was admitted to a rehabilitation facility with trophic ulcer on his left heal and inability to ambulate. His blood pressure on admission was consistent 80/50 and he was on midodrine 10 mg three times daily. The pressure sore on his left heal was non healing over 3 months despite pressure relief and local wound care. Patient was started on Aricept (donepezil) 5 mg daily. Over the course of 7 days the dose of midodrine was decreased to 5 mg daily and his blood pressure improved to 90/60. The ulcer on his left foot was showing improvement. The dose of donepezil was continued at 5 mg daily and over the next 10 days his blood pressure improved to 100/70. Midodrine was discontinued with no deterioration in his blood pressure. Patient tolerated the donepezil well and manifested no symptoms of angina, hypotension, or congestive failure. He was maintained on donepezil over the next two months with blood pressures measured 120-111.0/70-60 while on hemodialysis.

Ms. D. P. is an 81 year old female with history of right above knee amputation and severe coronary artery disease. Her ejection fraction was measured at 30%. Patient was admitted to a rehabilitation facility for gait training with a prosthesis. Her admission medications included Micardis 80 mg, Cardizem CD 240 mg daily and Imdur (long acting nitrate) 30 mg daily for management of angina, blood pressure and congestive failure. Cardiology consult upon admission noted failure with orthopnea and diuretic and lisinopril were started. Despite this treatment patient was dyspnic while supine and or exertion. Patient was started on Aricept (donepezil) 5 mg daily. Over the course of the next week Micardis was discontinued, and the dose of Cardizem reduced to 120 mg daily. Her symptoms of congestive heart failure resolved and the diuretic was discontinued. Her blood pressure normalized and, in the absence of exertional shortness of breath, the imdur and lisinopril were discontinued. Patient was able to engage in therapy using an above knee prosthesis which requires 60% more energy for ambulation.

Ms. B. W. is a 72 year old female with history of osteoporosis, congestive heart failure, with intermittent angina. She required 3 pillows for sleep secondary to nocturnal dyspnea while in the supine position. Her daily dose of furosemide (diuretic) was 60 mg daily for management of congestive failure. In addition to diuretic therapy she required long acting nitrates and intermittent sublingual nitroglycerin for shortness of breath associated with substernal chest pain or angina. Patient was started on Aricept (donepezil) 5 mg daily for management of dyspnea, anginal symptoms and lower extremity edema resulting from coronary disease/angina. Over the first week her diuretic requirement were lowered to 40 mg daily with improvement in lower extremity edema/swelling. After an additional seven days the dose of long acting nitrates (imdur) was reduced from 60 to 30 mg. During this time her diastolic blood pressure was less than 80 mm Hg. Over the next two weeks her diastolic blood pressure fell further to 70 mm Hg necessitating further reduction in furosemide to 20 mg every day, while no increase in lower extremity swelling developed. During this time there were no symptoms of congestive heart failure and the number of pillows required for elevation of the head was reduced to a single pillow. With no symptoms of exertional dyspnea, congestive heart failure and a blood pressure consistently 110/70 the long acting nitrate was discontinued without incident. The patient remained on 5 mg donepezil orally for over three years with no further requirements for nitrates or diuretic therapy.

Ms. O. W. is a 71 year old female with history of left above knee amputation, severe coronary artery disease (ejection fraction less than 30%) and congestive failure. Patient suffered from shortness of breath while supine requiring three pillows for partial relief of her symptoms. This was coupled with treatment using furosemide in doses of 40 mg daily. The patient was started on Aricept (donepezil) 5 mg daily. After one week of treatment she reported no further episodes of shortness of breath. There was a drop in her blood pressure to 100/60 during treatment. The dose of furosemide was decreased to 20 mg every other day. Follow up one week later confirmed no increase in shortness of breath and blood pressure improved to 110/70 without orthopnea. Patient was maintained on donepezil 5 mg daily for one month with no reported shortness of breath, or chest pain. Blood pressure normalized following cessation of diuretic therapy. She experienced no untoward side effects during treatment.

Ms. B. B. is a 62 year old female with history of coronary disease, morbid obesity, with marginal blood pressure requiring midodrine 5 mg orally three times daily to maintain blood pressure. Patient suffered from intermittent symptoms of congestive failure with recumbent dyspnea. The patient had undergone gastric by-pass surgery secondary to morbid obesity. Patient was admitted to a rehabilitation following bilateral total knee replacement. Patient was treated with daily diuretic therapy for management of lower extremity edema pre and post total knee replacement. Patient's blood pressure on admission was marginal despite hematocrit greater than 30 and midodrine 5 mg three times daily. Patient suffered from dyspnea consistent with congestive heart failure while supine. Patient was started on Aricept (donepezil) 5 mg daily. Over the next five days diuretic therapy was discontinued with normal blood pressure and no further episodes of shortness of breath. Over the next five days patient was able to discontinue midodrine with blood pressure greater than 100/60. Patient was discharged to home on donepezil 5 mg daily and follow up one month later revealed blood pressure 120/60 with no episodes of shortness of breath, symptoms of congestive heart failure and evidence of improved cardiac output.

### Metabolic Syndrome

The following case studies show the treatment of metabolic syndrome

Mr. S. W. is a 46 year old male with history of obesity, elevated triglycerides (greater than 400 mg %), and hypertension (BP approx. 180/95). Fasting blood sugars were greater than 140 mg % consistently. The patient was started on Aricept (donepezil) 5 mg daily. Measurement of fasting blood sugars was consistently less than 100 mg % during treatment. After one month of treatment with donepezil the patient's blood pressure was consistently less that 130/80, and follow up measurement of triglycerides were less than 180 mg %. Patients HDL cholesterol increased from 30 to 50 during this time.

Ms. K. E. is a 57 year old female with history of elevated triglycerides measured at greater than 200 mg %, HDL less than 40, hypertension and borderline diabetes status post left total knee replacement. She suffered from significant left lower extremity edema post operative, requiring diuretics for management but with unsatisfactory results. The patient was begun on Aricept (donepezil) 5 mg daily and followed after one week. There was notable decrease in left lower extremity edema and her need for blood pressure medications significantly reduced. The dose of imdur was reduced from 60 to 30 mg and the dose of amlodipine eliminated completely. After a second week of treatment the patient was taken off hydralazine and her blood pressure was maintained in an acceptable range. After one month of treatment her lipid profile was measured and found to have improved HDL cholesterol greater than 50 and triglycerides reduced from greater than 200 to less than 140 mg %. The patient maintained an acceptable blood pressure and marked reduction in left lower extremity edema.

Ms. M. L. is a 73 year old female with history of borderline diabetes with elevated fasting blood sugars, triglycerides 171, HDL cholesterol 37 and hypertension. Patient had undergone a right total shoulder replacement two years earlier. Patient was placed on Aricept (donepezil) 5 mg orally daily. Follow up lipid profile one month later revealed HDL cholesterol 52 and triglycerides measured at 93. Blood pressure was measured 130/70 and fasting blood sugars were less than 100 mg % consistently. Patient suffered no untoward side effects from treatment with donepezil.

Mr. L. E. is a 68 year old male with history of borderline diabetes mellitus, bilateral above knee amputations, HDL cholesterol 37, triglycerides 162, and borderline hypertension. He was started on Aricept (donepezil) 5 mg daily. The patient was seen in follow up visit one month later and his lipid profile measured HDL cholesterol 48 and triglycerides 132 (a significant improvement). The patient had previously required 25 units of Lantus insulin for management of diabetes. Over the course of treatment with donepezil his insulin diminished to 5 units of glargine insulin at bedtime. Fasting blood sugars were less than 110 mg % consistently after donepezil treatment.

Ms. D. P. is a 74 year old female with history of normal pressure hydrocephalus and a constellation of clinical finding consistent with metabolic syndrome. Fasting blood sugars were consistently greater than 125 mg %, HDL cholesterol was 43, triglycerides 171, and hypertension treated with losartan 80 mg daily. Patient was started on Aricept (donepezil) 5 mg daily for one month. Follow up at one week into treatment revealed a systolic blood pressure less than 120 mm Hg. Losartan was discontinued with normal blood pressures. Follow up measurement of HDL cholesterol and triglycerides one month post treatment with donepezil was 54 and 129, respectively, a significant improvement in both. Her cognition was improved over the course of treatment. Fasting blood sugars were consistently less than 100 mg %.

### Cephalization of Body Mass

Ms. S. M. is a 41 year old female with no significant past medical history with the exception of seasonal allergies. She was placed on Aricept (donepezil) 2.5 mg three times a week. The patient noted over the course of two weeks an increase in her bust size from 33A to 34 C. During the course of treatment she noted no untoward side effects from treatment and maintained same bust size on biweekly treatment with 2.5 mg donepezil.

Ms. A. T. is a 52 year old female with history of low back pain. Patient was started on Aricept (donepezil) 5 mg daily. Over the course of one month's treatment patient noted enlargement in breasts and increase in tone and cup size. During this time there was simultaneous cephalization of body mass with loss of abdominal girth and waist and hip size with redistribution of soft tissue over the clavicle and shoulder along with toning and enlargement in cup size. Patient suffered no untoward side effects from treatment and reported decrease in low back pain. Over the course of 4 months weight loss occurred in the range of 20 pounds (about 8.5 kg) and that weight loss stabilized despite continued treatment an additional two months.

Ms. M. J. is a 54 year old female with history of asthma, myofascial pain syndrome started on Aricept (donepezil) 5 mg daily. After two months of treatment patient noted improved tone of breasts and enlargement in cup size and bust measurement. There was concomitant redistribution and cephalization of body mass. Patient noted a loss of girth in hips and abdomen. Patients cup size increased by one size and one inch was added to breast measurement. Patient reported weight loss of 15 pounds (about 7 kg) in the absence of dieting.

Ms. B. B. is a 62 year old female with history of bilateral lower extremity lymphedema, and gastric by pass surgery. Five years following bariatric surgery patient was left with loss of soft tissue in the distribution of the neck, shoulders and breasts. She had enormous soft tissue distribution in the areas of hips and thighs. Patient was started on Aricept (donepezil) 5 mg daily. Over the course of three months time patient reported shrinkage of thighs and hip girth concomitant with cephalization of body mass in the distribution of neck, shoulders and breasts. Simultaneous with this redistribution of body mass patient noted over all weight loss of greater than 20 pounds (about 9 kg) in the absence of dieting and exercise program.

### Treatment of HIV Infection

Ms. M. B. is a 52 year old female with history of HIV infection treated with antivirals. She had a history of asthma, avascular necrosis left hip, and chronic pain. Patient had persistent of viral loads 1-2 million and CD4 counts less than 20. Patient was placed on Aricept (donepezil) 5 mg orally daily for one month. During that time she reported improvement in asthma symptoms and pain. Follow up measurement of viral load was 1 million and CD4 count 26. Patient continued on 5 mg daily of donepezil and CD4 and viral loads were measured following two months of treatment. Viral load following two months of treatment were non existent (below measurement detection) and CD4 count was 42. Patient suffered no infections or fevers during this time. She suffered no untoward side effects from treatment with donepezil.

Mr. B. D. is a 62 year old male with history of COPD (emphysema), osteoarthritis and HIV infection. Patient had sustained weight loss over the past 3 months of twenty pounds despite treatment with antivirals in therapeutic doses. Patient was started on Aricept (donepezil) 5 mg daily. Prior to treatment CD4 count was 24 and viral load over 4 million. Measurement of CD4 and viral loads after two months of treatment revealed improvement in CD4 count to 36 and reduction in viral load to 1.6 million. During this time patient gained over 15 pounds and reported improvement in pulmonary symptoms. He manifested no fevers, infections or untoward side effects from treatment with donepezil.

### EXAMPLE 1

This example provides a rough calculation of the hemoglobin A1c (HbA_{1C}) levels of the foregoing patients who each received donepezil according to each case study described above. The table below indicates the initial HbA_{1C} level prior to donepezil treatment and a follow up HbA_{1C} level and the time elapsed between HbA_{1C} measurements. While these data are based on a collection of patients who were treated off-label, it was not a controlled clinical trial, just an open label use of an available drug that has acetyl cholinesterase inhibiting properties.

TABLE 1

| | HbA_{1C} | HbA_{1C} after | Time between | % age |
|---|---|---|---|---|
| Patient initials | baseline | donepezil | measurements | reduction |
| J C | 6.4 | 4.6 | One month | 28.125% |
| J S | 11.8 | 9.1 | Five weeks | 22.88% |
| S H | 8.8 | 5.8 | One month | 34.10% |
| M M | 5.8 | 5.0 | One month | 13.79% |
| Q P | 7.3 | 5.8 | One month | 20.55% |
| B W | 9.1 | 6.8 | One month | 25.27% |
| E S | 9.3 | 6.3 | Two months | 32.26% |
| B L | 8.2 | 5.9 | Two months | 28.05% |
| J R | 9.5 | 6.8 | One month | 28.42% |
| W W | 6.4 | 5.8 | One month | 9.38% |
| M F | 11.5 | 7.9 | One month | 31.30% |
| Average (all) | | | | 24.91% |
| Average (above | | | | 26.03% |
| 6.0 initial) | | | | |

The case study data provided show an overall average percent reduction (after mostly one month but up to two months of treatment) of 24.91% (highly significant) with each patient serving as his or her own control. The lowest percentage reductions were for those patients having low initial HbA_{1C} levels. When one patient with an initial HbA_{1C} of 5.8 is removed (MM), the average percent reduction is 26.03%.

For comparison purposes, Transition Therapeutics announced on 28 Jun. 2007 that a phase 2 controlled clinical trial of a gastrin-based therapy showed a “significant reduction” in HbA_{1C} levels. Specifically, after one month of treatment the mean HbA_{1C} level reduction among treated patients was 0.43% after one month (not significant) and 0.93% after two months (significant). Their placebo-treated patients showed around a 0.1% increase or decrease in HbA_{1C} levels. Transition Therapeutics though these data were significant enough to warrant pivotal phase 3 Accordingly, treatment with donepezil, galantamine, rivastigmine, tacrine, and combinations thereof produced much more dramatic reductions of HbA_{1C} levels than have been seen before.

### EXAMPLE 2

This example provides a protocol for a double blind study of the acetyl cholinesterase inhibitor rivastigmine. A 12 week double blind randomized placebo controlled trial with increase in the dose of medication after one week of treatment such that there is one placebo group, one group receives 1.5 mg of rivastigmine twice daily and a third group receives 3.0 mg rivastigmine twice daily. The primary objective is to assess pain using patient questionnaire of their pain (VAS, visual analog scale) at weekly intervals. Upon completion of the 12 week trial the same questionnaire will be administered via examiner. Secondary objectives include a measure of sural nerve conduction, BUN (blood urea nitrogen), HbA_{1C} (hemoglobin A1C) and lipid profile to ascertain effect of cholinesterase inhibitor upon sensory nerve conduction, renal function and diabetic glycemic control and lipids respectively.

The initial dose of rivastigmine (Exelon) will be 1.5 mg twice daily. After one week the “dose” will be increased in the control group. The second group will have a “placebo” increase and the third group will have the dose increased to 3 mg twice daily in the absence of adverse side effects. There will be a total of 90 patients in the study with 30 in each group.

The inclusion criteria are all diabetic patients presenting to the Washington Hospital Center and the National Rehabilitation Hospital experiencing troublesome symptoms of diabetic polyneuropathy will be eligible. Exclusion criteria are pregnancy or lactation and children under 18 years of age. Patients taking other medications for diabetic neuropathy will be weaned from these medications prior to entering into the study.

Patients will be contacted weekly to assess their pain using VAS. Pre and Post studies of a more objective nature will include lipid profile, BUN, creatinine, and hemoglobin A1C and sural n conduction studies. All groups will have their dosage of medication titrated upward (doubled) after one week of therapy if no side effects noted.

Patients will be contacted weekly and asked to assess their average pain on a 10 point Likert scale. In addition to reporting pain patients will be asked if any adverse side effects were noted including blood pressure, gastrointestinal complaints, and blood glucose levels. Prior to onset of administration of the drug or placebo Sural Nerve conduction, Hemoglobin A1C, lipid profile and Blood Urea Nitrogen/creatinine will be measured. After completion of the twelve week trial follow up nerve conduction and laboratory studies will be obtained to assess potential benefit to blood glucose control and renal function.

The initial dose of rivastigmine (Exelon) will be 1.5 mg twice daily. After one week the “dose” will be increased in the control group. The second group will have a “placebo” increase and the third group will have the dose of rivastigmine increased to 3.0 mg twice daily in the absence of adverse side effects. There will be a total of 90 patients in the study with 30 in each group.

### EXAMPLE 3

This example illustrates a combination therapy of an acetylcholinesterase inhibitor (preferably donepezil) has significant efficacy for glycemic control and reducing or eliminating the need for insulin when combined with metformin. In the case studies provided above, the best reduction in HbA_{1C} levels was patient “SH.” SH had a history of Insulin Dependent Diabetes Mellitus admitted to a rehabilitation facility following lumbar laminectomy for stenosis. Upon admission her diabetic control was managed with 12 units of Lanuts (glargine) insulin at bedtime. Her HbA_{1C} was measured on admission and found to be 8.8, indicating poor blood sugar management. The patient was started on Aricept (donepezil) 5 mg orally daily and amaryl 4 mg and metformin 500 mg twice daily. The patient tolerated the dose of 5 mg orally of donepezil daily and after one week the dose was increased to 10 mg orally at bedtime. The HbA_{1C} was measured one month later and found to have improved to 5.8. During this time her requirements for insulin decreased and she no longer required lantus insulin for control of her blood sugars. When she was started on donepezil the lantus was discontinued. Within 48 hours of increasing the dose of donepezil to 10 mg patient discontinued amaryl and was maintained on metformin 500 mg orally twice daily. While the metformin was later discontinued, the initial combination of donepezil and metformin allowed this patient to discontinue insulin and to significantly improve her glycemic control.

Therefore, the present disclosure provides a method for treating diabetes, including long term complications of diabetes and glycemic control, comprising administering a formulation comprising an acetyl cholinesterase inhibitor and from about 200 mg to about 1000 mg of metformin (once or twice daily). Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the twice daily dose of metformin is from about 400 mg to about 1000 mg.

The present disclosure further provides a method for reducing necessary doses of insulin or other glycemic control medications comprising administering a formulation comprising an acetyl cholinesterase inhibitor and from about 200 mg to about 600 mg of metformin (once or twice daily). Preferably, the acetyl cholinesterase inhibitor compound is selected from the group consisting of donepezil, galantamine, rivastigmine, tacrine, combinations thereof, and pharmaceutically acceptable salts thereof. Most preferably, in an adult the daily dosage of donepezil is from about 5 mg to about 10 mg, the daily dosage of galantamine is from about 16 mg to about 32 mg, and the daily dosage of rivastigmine is from about 3 mg to about 9 mg. Preferably, the twice daily dose of metformin is from about 400 mg to about 1000 mg.

I claim:

1. A method for glycemic control of a patient having a disease selected from the group consisting of type 1 diabetes mellitus, type 2 diabetes mellitus, impaired glucose tolerance, hyperglycemia, and postprandial hyperglycemia, said method comprising administering to a patient in need thereof a pharmaceutical composition comprising an acetyl cholinesterase inhibitor compound.

2. The method for glycemic control of a patient having a disease selected from the group consisting of type 1 diabetes mellitus, type 2 diabetes mellitus, impaired glucose tolerance, hyperglycemia, and postprandial hyperglycemia of claim 1, wherein the acetyl cholinesterase inhibitor compound is selected from the group consisting of donepezil, galantamine, rivastigmine, tacrine, combinations thereof, and pharmaceutically acceptable salts thereof.

3. The method for glycemic control of a patient having a disease selected from the group consisting of type 1 diabetes mellitus, type 2 diabetes mellitus, impaired glucose tolerance, hyperglycemia, and postprandial hyperglycemia of claim 2, wherein in an adult the daily dosage of donepezil is from about 5 mg to about 10 mg, the daily dosage of galantamine is from about 16 mg to about 32 mg, and the daily dosage of rivastigmine is from about 3 μg to about 9 mg.

4. A method for reducing hemoglobin A1C concentrations as a measure of glycemic control, comprising administering to a patient in need thereof a pharmaceutical composition comprising an acetyl cholinesterase inhibitor compound.

5. The method for reducing hemoglobin A1c concentrations as a measure of glycemic control of claim 4, wherein the acetyl cholinesterase inhibitor compound is selected from the group consisting of donepezil, galantamine, rivastigmine, tacrine, combinations thereof, and pharmaceutically acceptable salts thereof.

6. The method for reducing hemoglobin A1C concentrations as a measure of glycemic control of claim 5, wherein in an adult the daily dosage of donepezil is from about 5 mg to about 10 mg, the daily dosage of galantamine is from about 16 mg to about 32 mg, and the daily dosage of rivastigmine is from about 3 mg to about 9 mg.

7. A pharmaceutical formulation for daily administration comprising an acetyl cholinesterase inhibitor, from about 5 mg to about 15 mg of loratadine, from about 5 mg to about 16 mg of elemental zinc, or from about 10 mg to about 50 mg per day dose of metaclopramide.

8. The pharmaceutical formulation for daily administration of claim 7 wherein the acetyl cholinesterase inhibitor compound is selected from the group consisting of donepezil, galantamine, rivastigmine, tacrine, combinations thereof, and pharmaceutically acceptable salts thereof.

9. The pharmaceutical formulation for daily administration of claim 8 wherein in an adult the daily dosage of donepezil is from about 5 mg to about 10 mg, the daily dosage of galantamine is from about 16 mg to about 32 mg, and the daily dosage of rivastigmine is from about 3 mg to about 9 mg.

10. The pharmaceutical formulation for daily administration of claim 7 wherein the daily dose of loratadine is from about 8 mg to about 12 mg.

11. The pharmaceutical formulation for daily administration of claim 7 wherein the elemental zinc daily does is from about 8 mg to about 13 mg.

12. A method for treating diabetes comprising administering a formulation comprising an acetyl cholinesterase inhibitor, from about 5 mg to about 1.5 mg of loratadine, optionally from about 5 mg to about 16 mg of elemental zinc, and optionally from about 10 mg to about 50 mg per day dose of metaclopramide.

13. The method for treating diabetes of claim 12, wherein the acetyl cholinesterase inhibitor compound is selected from the group consisting of donepezil, galantamine, rivastigmine, tacrine, combinations thereof, and pharmaceutically acceptable salts thereof.

14. The method for treating diabetes of claim 13, wherein in an adult the daily dosage of donepezil is from about 5 mg to about 10 mg, the daily dosage of galantamine is from about 16 mg to about 32 mg, and the daily dosage of rivastigmine is from about 3 mg to about 9 mg.

15. A method for treating diabetes, including long term complications of diabetes and glycemic control, comprising administering a formulation comprising an acetyl cholinesterase inhibitor, from about 200 mg to about 600 mg of metformin (once or twice daily), and optionally from about 10 mg to about 50 mg per day dose of metaclopramide.

16. The method for treating diabetes, including long term complications of diabetes and glycemic control of claim 15 wherein the acetyl cholinesterase inhibitor compound is selected from the group consisting of donepezil, galantamine, rivastigmine, tacrine, combinations thereof, and pharmaceutically acceptable salts thereof.

17. The method for treating diabetes, including long term complications of diabetes and glycemic control of claim 16 wherein in an adult the daily dosage of donepezil is from about 5 mg to about 10 mg, the daily dosage of galantamine is from about 16 mg to about 32 mg, and the daily dosage of rivastigmine is from about 3 mg to about 9 mg.

18. The method for treating diabetes, including long term complications of diabetes and glycemic control of claim 15 wherein the twice daily dose of metformin is from about 400 mg to about 600 mg.

19. A method for treating coronary artery disease, congestive heart failure or cardiomyopathy in a patient comprising administering to a patient a pharmaceutical composition comprising an acetyl cholinesterase inhibitor compound.

20. The method for treating coronary artery disease, congestive heart failure or cardiomyopathy of claim 19 further comprising administering from about 5 mg to about 15 mg of loratadine, optionally from about 5 mg to about 16 mg of elemental zinc, and optionally from about 10 mg to about 50 mg per day does of metaclopramide.

21. A method for treating obesity and promoting cephalization of body mass, comprising administering to a patient a pharmaceutical composition comprising an acetyl cholinesterase inhibitor compound.

22. A method for treating metabolic syndrome to prevent its progression to diabetes, the side effects of corticosteroid administration or restless leg syndrome, comprising administering to a patient a pharmaceutical composition comprising an acetyl cholinesterase inhibitor compound.

23. A method for treating HIV infection in an infected patient, or for treating cancer by trophic stimulation of neoplastic cancer cells to promote normal growth and function, comprising administering to the infected patient a pharmaceutical composition comprising an acetyl cholinesterase inhibitor compound.
